(19) United States
(12) Patent Application Publication
(10) Pub. No.: US 2017/0153180 A1
(43) Pub. Date: Jun. 1, 2017

(54) COMPOSITIONS, METHODS AND DEVICES THEREOF FOR FLUORESCENT ANALYSIS OF GUNSHOT RESIDUE

(71) Applicant: DUQUESNE UNIVERSITY OF THE HOLY GHOST, PITTSBURGH, PA (US)

(72) Inventors: Partha Basu, Pittsburgh, PA (US); Antoinette Peterson, Pittsburgh, PA (US); John Thomas, Canfield, OH (US)

(73) Assignee: DUQUESNE UNIVERSITY OF THE HOLY GHOST, PITTSBURGH, PA (US)

(21) Appl. No.: 15/325,538

(22) PCT Filed: Jul. 31, 2015
(86) PCT No.: PCT/US15/43052

§ 371 Date: Jan. 11, 2017

Related U.S. Application Data
(60) Provisional application No. 62/031,281, filed on Jul. 31, 2014.
(60) Provisional application No. 62/054,601, filed on Sep. 24, 2014.

(51) Int. Cl. G01N 21/64; G01N 33/20

(52) U.S. Cl. CPC G01N 21/643; G01N 21/6447; G01N 33/20; G01N 2021/6439

(57) ABSTRACT

The present disclosure relates to lead ion sensors for testing, detecting and analyzing particle-containing samples for gunshot residue. The lead ion sensors include a fluorophore itself or a combination of a fluorophore and matrix material. The particle-containing samples are contacted with the lead ion sensors. Due to a presence of Pb²⁺ ions in a sample, a fluorescence emission is visually observed and a correlation of a presence of gunshot residue can be made. In addition, the fluorescence emission intensity can be assessed to obtain information relating to the GSR sample. Further, there is provided a device for conducting on-site testing, detecting and analyzing of particle-containing samples for gunshot residue.

### CROSS REFERENCE TO RELATED APPLICATIONS

This application claims priority to U.S. Provisional Application Ser. Nos. 62/031,281, entitled “Compositions and Methods Thereof For Use in Fluorescent Analysis of Gunshot Residue”, filed on Jul. 31, 2014, and 62/054,601 entitled “Methods and Devices for Fluorescent Analysis of Gunshot Residue”, filed on Sep. 24, 2014, the disclosures of which are incorporated in their entirety by this reference.

### FIELD OF THE INVENTION

The invention relates to compositions and methods for the detection and analysis of lead ions in gunshot residue and, in particular, to fluorophores and their use as sensors for detecting lead ions based on fluorescence emission of gunshot residue and, more particularly, to portable devices for on-site detection and analysis.

### BACKGROUND

Gunshot residue (GSR) analysis can be instrumental in investigating situations involving a firearm. Generally, when a firearm is discharged, residue deposits on the body and/or clothes of the individual firing the firearm, as well as on surfaces or persons nearby. Analysis of the residue has important forensic application in identifying the individual who fired the firearm and persons near the firearm when it was discharged.

GSR analysis has become a significant branch of forensic science. Traditional GSR analysis methods typically involve the use of scanning electron microscopy coupled with energy dispersive X-ray for elemental analysis (SEM-EDX), or chromatographic separation coupled with electrophoresis and/or mass spectrometry. These methods employ complex apparatus and techniques. In general, gunshot residue samples are collected, such as, by crime scene investigators using adhesive lifters to obtain a sampling of the GSR, and transported to a laboratory for analysis. Traditional analysis of the sample includes searching, for particles with characteristic morphology of GSR and performing an elemental analysis without destroying the particles.

Traditional GSR analysis methods are useful for confirming the presence of pruner GSR and therefore, as previously indicated, can be effective to determine if a person was holding a firearm and if a person as near a firearm at the moment it was discharged. However, the methods do not have the capability to provide additional information. That is, due to their purely indicative nature, the methods cannot distinguish between a person who fired the weapon in question, or a person who was merely near the weapon when it was fired. Secondary transfer of GSR is facile and therefore, persons not even at the scene where the weapon was discharged can have GSR on their hands if they are in contact with the weapon or merely in contact with the hand(s) of the person who actually fired the weapon.

There are advantages associated with traditional GSR analysis methods, such as, studies have shown that traditional collection of gunshot residue samples is generally resistant to contamination from evidence collectors improperly handling the collection stub and container, which means that police officers or other investigators who are not necessarily scientists can collect the evidence without compromise. This is important, as early collection is needed to avoid the risk that the subject will wash his or her hands or clothing and prevent the detection of GSR. However, there are also disadvantages associated with traditional GSR analysis methods, such as, being instrument intensive and requiring trained analysts to search for GSR particles over the sample stub, which may be tedious, as well as performing elemental analysis that may lead to inconclusive results.

Thus, there is a need in the art to develop new methods of GSR analysis that are less instrument intensive, easier to perform and evaluate, and capable of providing conclusive results. Further, it would be advantageous if the methods and associated instrumentation were portable, such that the GSR analysis may be conducted and results obtained outside a laboratory, such as, at the scene where the firearm was discharged. In accordance with the invention, fluorescent molecules are utilized to perform GSR analysis.

When the firearm is discharged, gaseous particles are released from any part of the gun that is not air-tight. These gaseous particles form a plume that is so concentrated that some particles hit each other and stick together, so that when the vapor sublimes, there are particles that contain all three metals from the gunshot primer: lead, barium and antimony. There are also particles that contain only one or two of these metals. These particles are not considered characteristic of GSR. These particles can be classified as either consistent or commonly associated with GSR, respectively.

With the exception of some new nontoxic products, all ammunition contains lead. Thus, a sensitive, measurement of lead in GSR can provide direct evidence useful in a firearm investigation.

Historically, other methods have been used to detect trace metals, namely atomic absorption (AA) and inductively coupled plasma mass spectrometry (ICP-MS). Both methods have exemplary sensitivity in detection of trace metals. There are standard methods validated by the Environmental Protection Agency (EPA) for analysis of trace metals in water, due to the severe human health and environmental effects of lead poisoning. In addition to ICP-MS and graphite furnace atomic absorption (GF-AA), the EPA has also validated a method using inductively coupled plasma atomic emission spectrometry (ICP-AES). The limits of detection reported for EPA methods for quantifying lead in solution are shown below.

| Method | Detection Limit (μg/L) |
|---|---|
| ICP-MS | 0.05 |
| (selection ion monitoring, | |
| total recoverable) | |
| ICP-AES | 1.1 |
| GF-AA | 0.7 |

The above-mentioned methods are advantageous over scanning electron microscopy (SEM) because they are more sensitive and therefore, more likely to detect smaller amounts of lead. Further, they can be used for quantitation, in addition to qualitative analysis, which is another benefit when compared to SEM. However, a disadvantage of these methods is that they require the use of an aqueous sample, and GSR particles are solid. When GSR particles are placed into solution, typically using an acid such as 5-10% nitric acid it has been found that the different metals, namely lead, barium and antimony, are separated from each other. They are then three separate components of a general mixture, instead of three components of an individual particle. This separation decreases discrimination power and as a result, these methods are not useful as confirmatory tests for GSR. Thus, the preferred method in the art is SEM-EDX, which does not require liquid samples and involves minimal sample preparation. However, it can take significantly longer lengths of time to run SEM-EDX. Due to the slow throughput, there can be a backlog for SEM analysis of GSR. Thus, in accordance with the invention, the detection of lead in GSR using fluorescent-based lead ion detection provides a quicker and earlier screening test, and can limit the number of samples analyzed by SEM by eliminating definite negative samples.

Traditionally, any presumptive, color tests for GSR are not generally used. One significant problem is that these tests are not selective enough to justify using them as a screening tool. The first colorimetric test for GSR was the Dermal Nitrate Test or Paraffin Test (in 1933). This test was used to detect nitro groups that were present on an offender's hand following enacting the discharge of a firearm. However, it is known that this test gives false positives since nitro groups are ambiguous to the environment. In one study, about half the subjects who did not fire a weapon tested positive for GSR using the Paraffin test. Another difficulty with traditional colorimetric tests for GSR is that none are sensitive enough to detect a trace amount of residue with low risk for false negatives. Sodium rhodizonate has been used historically in GSR tests, particularly in the Harrison-Gilroy test. It is the only color test still commonly used for analysis of GSR. Although, the Harrison-Gilroy test: is usually used for bulk samples such as short to medium distance gunshot wounds. This is because, while sodium rhodizonate is sensitive enough to detect the larger amounts of lead that are deposited on the skin of a shooting victim, it is not sensitive enough to detect the residue that is deposited onto the hands of the shooter.

The use of fluorescent metal probes and portable fluorometry methods can provide advantages in the field of forensic science and the analysis of GSR.

In general, fluorescent molecules have various uses, for example, but not limited to, in labeling and detection of substrates or molecules in cell based assays, as components in organic electronic materials in molecular electronics, as pH sensors, and as metal sensors. There are currently several general classes of fluorescent molecules. These have been divided based on their structural motifs. For example, some common fluorescent structures include xanthene based fluorescein and rhodamine compounds, coumarins, pyrenes, and molecules based on the cyanine dyes. Other common fluorophores include, for example, auramine, acridine orange, dipyrrin, and porphyrin.

Molecular fluorescence is a type of photoilluminescence, which is a chemical phenomenon involving the emission of light from a molecule that has been promoted to an excited state by absorption of electromagnetic radiation. Specifically, fluorescence is a luminescence in which the molecular absorption of a photon triggers the emission of a second photon with a longer wavelength (lower energy) than the absorbed photon. The energy difference between the absorbed photon and the emitted photon results from an internal energy transition of the molecule where the initial excited state (resulting from the energy of the absorbed photon) transitions to a second, lower energy excited state, typically accompanied by dissipation of the energy difference in the form of heat and/or molecular vibration. As the molecule decays from the second excited state to the ground state, a photon of light is emitted from the compound. The emitted photon has an energy equal to the energy difference between the second excited state and the ground state.

Many fluorescent compounds absorb photons having a wavelength in the ultraviolet portion of the electromagnetic spectrum and emit light having a wavelength in the visible portion of the electromagnetic spectrum. However, the absorption characteristics of a fluorophore are dependent on the molecules absorbance curve and Stokes shift (difference in wavelength between the absorbed and emitted photon), and fluorophores may absorb in different portions of the electromagnetic spectrum.

The basic structures of known fluorophores may be modified to provide different excitation and emission profiles. For example, two Mated compounds, fluorescein and rhodamine have different fluorescent characteristics, fluorescein absorbs electromagnetic radiation having a wavelength of ˜494 nanometers (“nm”) and emits light having a wavelength at ˜525 nm, in the green region of the visible spectrum, whereas rhodamine B absorbs is radiation having a wavelength of ˜510 nm and emits light with an emission maximum of ˜570 nm, in the yellow-green region of the visible spectrum. Other fluorophores have different absorption and emission profiles. For example, coumarin-1 absorbs radiation at 360 nm and emits light at ˜460 nm (blue light); and pyrene absorbs radiation at ˜317 nm and emits light having a wavelength of ˜400 nm (violet light).

It is contemplated that the use of a fluorescent-based method can provide a quick, simple means of detecting lead ions and therefore, GSR. Additionally, this method can be portable since it does not require the use of complex equipment and techniques, and therefore can be employed at the scene where the firearm was discharged, e.g., at the scene of a crime or accident. Analysis of gunshot residue can be conducted in the absence of a laboratory analysis (e.g. SEM/EDS) or in conjunction with a laboratory analysis, e.g., for use to obtain preliminary results prior to a laboratory analysis.

### SUMMARY OF THE INVENTION

Various embodiments provide for fluorescent compounds that are capable of detecting the presence of Pb²⁺ in particle-containing gunshot residue samples. Other embodiments relate to methods and uses of the fluorescent compounds as detectors for Pb²⁺ in gunshot residue based on fluorescence emission. Still, other embodiments relate to devices for on-site testing of particle-containing samples for gunshot residue.

In one aspect, the present disclosure provides a sensor including a fluorescent binder for Pb²⁺ ions. The sensor is useful in the testing, detection and analysis of gunshot residue samples. The fluorescein binder is represented in its protected form by Formula II:

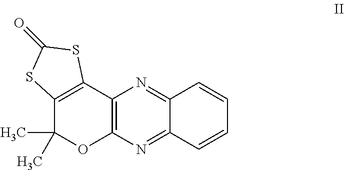 [SMILES: CC1(C)Oc2nc3ccccc3nc2-c2sc(=O)sc21]

The Pb²⁺ sensor for analyzing a particle-containing sample for gunshot residue includes a matrix material and a fluorophore represented in its protected form by Formula II, wherein the fluorophore is dissolved in, embedded in, affixed in, absorbed in, or suspended in Me matrix material and forms a fluorescent complex when bound in its unprotected form to Pb²⁺.

The matrix material can include a material selected from the group consisting of an aqueous solvent, a gel, a sol-gel material, a solvent, a paper, a polymer, a nanoparticle, a solid state material, and a surface-modified material.

The sensor can also include a device capable of measuring an intensity of a fluorescence emission spectrum.

In certain embodiments, one or more hydrogens on the fluorophore can be replaced with a group reactive with a functionality in the matrix materials.

In another aspect, the present disclosure provides a method of analyzing gunshot residue. The method includes contacting a particle-containing gunshot residue sample with a Pb²⁺ sensor comprising a fluorophore represented in its protected form by Formula II, wherein Pb²⁺ in the gunshot residue sample hinds with the fluorophore in its unprotected form to form a complex, and determining a presence or absence of fluorescence of the gunshot residue sample. The presence of fluorescence correlates to the presence of Pb²⁺ ions in the gunshot residue sample and the absence of fluorescence correlates to the absence of Pb²⁺ ions in the gunshot residue sample. Further, the method can include measuring a fluorescence emission intensity of the complex. Furthermore, the method may include calculating the concentration of Pb²⁺ ions in the gunshot residue sample based on the fluorescence emission intensity of the complex.

In certain embodiments, the method includes, establishing a threshold level based, on one of fluorescence emission intensity of the complex or concentration of Pb²⁺, comparing one of fluorescence emission intensity of the complex or concentration of for the gunshot residue sample with the threshold level, determining that the gunshot residue sample is a result of a direct transfer if the threshold value is met or exceeded, and determining that the gunshot residue sample is a result of a secondary transfer if the threshold value is not met.

In certain aspects, the disclosure also provides a portable device for detecting Pb²⁺ ions in a particle-containing sample for analysis of gunshot residue. The device includes a fluorophore sensor and, an activation source to excite a complex formed by the Pb²⁺ ions and the fluorophore sensor material, and to produce a fluorescence light output.

In certain aspects, the disclosure further provides a portable method of analyzing a gunshot residue sample. The method includes transporting a kit to a site of a discharged firearm. The kit includes a fluorophore sensor material and an illumination source. The method also includes obtaining a particle-containing sample to be analyzed, contacting the sample with the fluorophore sensor material, applying the illumination source to the sample with the fluorophore sensor material, visually observing whether the sample with the fluorophore sensor material fluoresces, determining a presence of Pb²⁺ ions wherein fluorescence is visually observed, and determining an absence of Pb²⁺ ions wherein fluorescence is not visually observed.

### BRIEF DESCRIPTION OF THE DRAWINGS

The various embodiments of the present disclosure will be better understood when read with reference to the following figures.

FIG. 1 illustrates a synthetic scheme for the synthesis of an intermediate for the preparation of the fluorophores according to the present disclosure.

FIGS. 2 and 3 illustrate synthetic schemes for generating fluorophores possessing structurally distinct formulas according to various embodiments of the present disclosure.

FIG. 4 is a plot showing lead amounts after firing a firearm for control samples and samples prepared in accordance with various embodiments of the present disclosure.

FIG. 5 is a plot showing lead amounts after firing a firearm for control samples and samples prepared in accordance with various embodiments of the present disclosure.

FIG. 6 is a plot showing emission spectra of standard lead solutions with varying amounts or fluorophore (i.e., designated LG) added, in accordance with various embodiments of the present disclosure.

FIG. 7 is a plot of maximum emission intensity, in accordance with various embodiments of the present disclosure.

FIG. 8 is a plot of fluorescence spectra for various samples containing equal amounts of fluorophore (i.e., designated LG).

### DETAILED DESCRIPTION

The present disclosure relates to fluorophores and, fluorophore and matrix composites as sensors to detect lead ions in a sample analyzed for gunshot residue (GSR). The disclosure also relates to methods and devices for testing, detecting and analyzing particle-containing samples for the presence of lead ions. Since GSR is at least partially composed of lead, the presence of lead ions in the sample is determinative of GSR. Further, the present disclosure relates to evaluating the fluorescence emission intensity of the sample to assess origin of the GSR, e.g., direct or secondary transfer.

The fluorophores may be synthesized from readily available materials. The structure of the fluorophores is designed with the flexibility to have multiple substitution patterns. The fluorophores can detect Pb²⁺ ions in gunshot residue samples and as a result of the fluorescence emission intensity, the level quantity of GSR in samples may be determined.

Other than, the operating examples, or where otherwise indicated, all numbers expressing quantities of ingredients, processing conditions and the like used in the specification and claims are to be understood as being modified in all instances by the term “about”. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical values, however, may contain certain errors, such as, for example, equipment and/or operator error, necessarily resulting from the standard deviation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, baying a minimum value equal to or greater than 1 and a maximum value of less than or equal to 10.

Any patent, publication, or Other disclosure, material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The present disclosure describes several different features and aspects of the invention with reference to various exemplary non-limiting embodiments. It is understood, however, that the invention embraces numerous alternative embodiments, which may be accomplished by combining any of the different features, aspects, and embodiments described herein in any combination that one of ordinary skill in the art would find useful.

The present disclosure relates to lead ion sensor fluorophores for analysis of GSR. The fluorophores have a structure comprising at least three fused rings including a five membered ring containing an ene-dithiolate moiety, a six-membered pyran ring, and a six-membered pyrazine ring. The general structure of the new class of fluorophores is represented by Formula I.

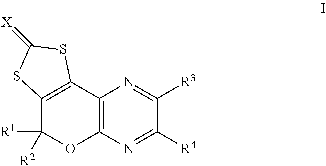

In Formula I, X represents a group such as O (i.e., a carbonyl, a “dithiolone”), S (i.e., a thiocarbonyl), Se (i.e., a selenocarbonyl), NR^{x} (i.e., an imine), NR^{x}₂⁺ (an iminium ion), or NNHR^{x} (a hydrazine). Each R^{x} may independently be a group such as hydrogen, the group -L-R^{y}, C₁-C₆ alkyl, phenyl, and substituted phenyl. The substituted phenyl may have from 1 to 5 substituents where each substituent may independently be one or more of the group -L-R^{y}, a fluoro, chloro, bromo, nitro, cyano, hydroxy, amino, thiol, C₃-C₆ alkyl, and C₁-C₆ alkoxy. As used herein, the term “Cₗ-C₆ alkyl” means an alkyl substituent having from 1 to 6 carbon atoms arranged either as a linear chain or as a branched chain. As used herein, the term “C₁-C₆ alkoxy” means an alkoxy substituent having from 1 to 6 carbon atoms arranged either as a linear chain or as a branched chain and attached in an ether linkage.

Further, in Formula I, R¹ and R² may each independently be hydrogen, the group -L-R^{y}, C₁-C₆ alkyl, amino C₁-C₆ hydroxy C₁-C₆ alkyl, thio C₁-C₆ alkyl, carboxyl C₁-C₆ alkyl, halo C₁-C₆ alkyl phenyl, substituted phenyl, aryl, substituted aryl, heteroaryl, or substituted heteroayrl. The substituted phenyl, aryl, or heteroaryl may have from 1 to 5 substituents where each substituent may he one or more of fluoro, chloro, bronco, nitro, cyano, hydroxy, amino, thiol, C₁-C₆ alkyl, amino C₁-C₆ alkyl, hydroxy C₁-C₆ alkyl, thio C₁-C₆ alkyl, carboxyl C₁-C₆ alkyl, halo C₁-C₆ alkyl, and C₁-C₆ alkoxy. As used herein, the terms “aryl” or “aryl ring” include an aromatic ring (i.e., a single aromatic ring) or ring system (i.e., a polycyclic aromatic ring system) in which all ring atoms are carbon. As used herein, the terms “heteroaryl” or “heteroaryl ring” include an aromatic ring (i.e., a single aromatic ring) or ring system (i.e., a polycyclic aromatic ring system) in which at least one of the ring atoms is a heteroatom, such as nitrogen, oxygen or sulfur heteroatom.

In Formula I, R³ and R⁴ may independently be the group -L-R^{y}, hydrogen, C₁-C₆ alkyl, amino C₁-C₆ alkyl, hydroxy C₁-C₆ alkyl, thio C₁-C₆ alkyl, carboxy C₁-C₆ alkyl, halo C₁-C₆ alkyl, phenyl, substituted phenyl, aryl, substituted aryl, heteroaryl, or substituted heteroaryl. The substituted phenyl, aryl, or heteroaryl may have from 1 to 5 substituents where each substituent may be one or more of a fluoro, chloro, bromo, nitro, cyano, hydroxy, amino, thiol, C₁-C₆ alkyl, amino C₁-C₄ alkyl, hydroxy C₁-C₆ alkyl, thio C₁-C₆ alkyl, carboxy C₁-C₆ alkyl, halo C₁-C₆ alkyl, and C₁-C₆ alkoxy. Alternatively in certain embodiments, R³ and R⁴ may come together to form one of a benzo ring, a substituted benzo ring, an aryl ring, a substituted aryl ring, a heteroaryl ring, or a substituted heteroaryl ring. The substituted benzo, substituted aryl, or substituted heteroaryl ring(s) may have from 1 to 4 substituents where each substituent may be one or more of the group -L-R^{y}, a fluoro, chloro, bromo, nitro, cyano, hydroxy, amino, thiol, C₁-C₆ alkyl, amino C₁-C₆ alkyl, hydroxy C₁-C₆ alkyl, thio C₁-C₆ alkyl, carboxy C₁-C₆ alkyl, halo C₁-C₆ alkyl, and C₁-C₆ alkoxy.

In preferred embodiments, the lead ion sensor fluorophore has a structure represented by Formula I, wherein X is O, R¹ and R² are each methyl, and R³ and R⁴ come together to form a benzo ring.

According to the various embodiments, the fluorophores of the present disclosure exhibit fluorescence, for example, when bound to Pb²⁺ ions. That is, the fluorophores of the present disclosure absorb electromagnetic radiation. Upon absorption of the electromagnetic radiation, the frontier electron (for single electron excitation) of the fluorophores is promoted to an excited electronic state which then decays to a second excited electronic state concomitant with molecular vibration and/or the release of heat. The fluorophores decay from the second excited state to the ground electronic state with the emission of electromagnetic radiation, wherein the emitted electromagnetic radiation has a wavelength that is longer than the wavelength of the absorbed radiation. For example, certain embodiments of the fluorophores having the structures set forth herein may absorb electromagnetic radiation having a wavelength within the ultraviolet region of the electromagnetic spectrum and fluoresce, that is emit electromagnetic radiation, at a wavelength within the blue light region of the visible spectrum. In certain embodiments, the fluorophores of the present disclosure may fluoresce with an emission maximum at a wavelength within the ultraviolet or visible regions of the electromagnetic spectrum. According to certain embodiments, the fluorophores of the present disclosure may fluoresce with an emission maximum at a wavelength from 200 nm to 850 nm. According to other embodiments, the emission maximum may be at a wavelength from 300 nm to 600 nm. According to other embodiments, the emission maximum may be at a wavelength from 400 rum to 500 nm. As used herein, the term “emission maximum” means the wavelength of the greatest intensity within the fluorescence spectrum of a fluorophore.

According to certain embodiments, the fluorophore includes a reactive group that can react with and form a bond to Pb²⁺ ions. The product of the chemical reaction between the fluorophore and Pb²⁺ ions will then fluoresce.

Under basic conditions, fluorescence of the fluorophores may be quenched when in the presence of certain transition metal ions. However, in the presence of other closed shell metal ions (such as Pb²⁺) the fluorescence of the fluorophores is still present. Significant fluorescence may be observed when fluorophore is complexed with Pb²⁺ ions, where the fluorescence is at a wavelength which is different from fluorescence wavelength observed from the other ions. For example, a Pb²⁺/fluorophore complex may fluoresce at a wavelength of ˜470 nm, whereas a Zn²⁺/fluorophore complex fluorescence shifts to a higher energy wavelength of ˜606 nm after 24 hours of incubation. The intensity of the fluorescent emission spectrum of the complex may be determined. According to specific embodiments, the fluorescent emission spectrum of the complex may be qualitatively used to determine the presence of a metal ion in a solution or, alternatively, may be quantitatively used (for example, by the intensity of the emission spectrum) to determine the concentration of the metal ion in the composition.

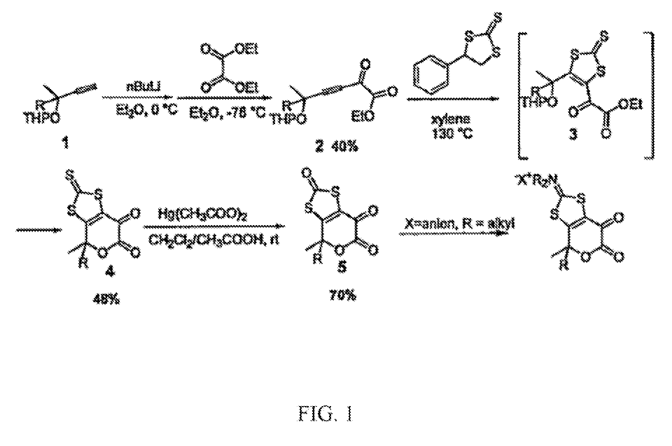

According to various embodiments, the fluorophores of the present disclosure may be readily synthesized using organic chemistry techniques. Preferred techniques are disclosed in U.S. Pat. No. 8,247,551 B2 (Basu, et al.). For example, as illustrated in FIG. 1 herein, the synthetic approach begins with the protection of the substituted propargyl alcohol with tetrahydropyran protecting group resulting in alkyne 1. The terminal alkyne in compound 1 is then deprotonated with n-butyl lithium and the reaction of the resulting acetylide with diethyl oxalate at a low temperature yields keto ester 2. The presence of an electron-withdrawing group (i.e., the ketone) activates the alkane functionality toward the reaction with styrene trithiocarbonate to introduce the protected dithiolene moiety. When the reaction is performed neat, the open intermediate 3 is isolated and then transformed to the pyran-dione 4 upon addition of trifluoroacetic acid. Conversely, when the reaction is performed in xylene, the pyran-dione 4 is isolated directly. Next, the dithiolethione functionality may be converted to the dithiolone by treating pyran-dione 4 with mercuric acetate. The resulting dithiolone 5 can be convened to an imine or iminium ion by reacting the dithiolone with an appropriate amine. As will be understood by one having ordinary skill in the art, any of compounds 4 or 5 may be converted to the fluorophore, thereby resulting in variations of X as shown in Formula I.

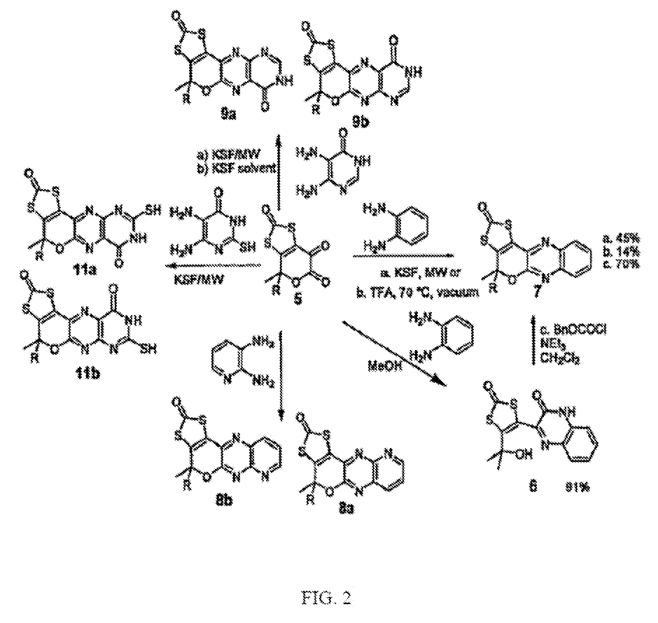

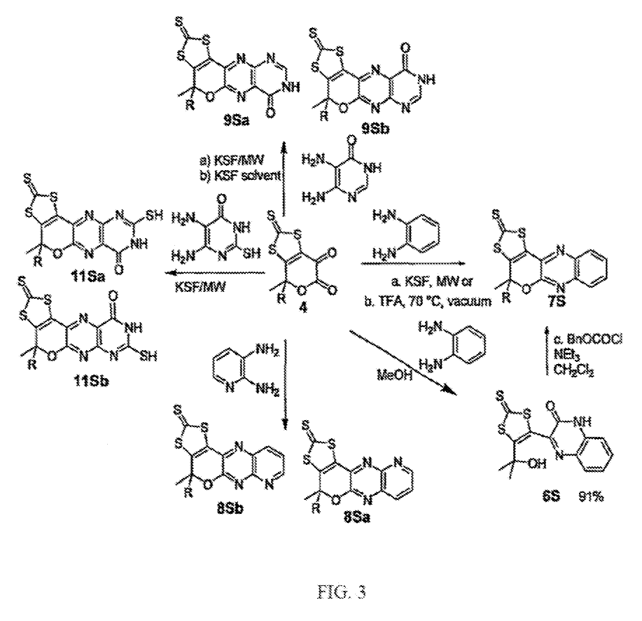

Once the diketo-compounds 4 or 5 are prepared they may be reacted with a variety of diamines to produce different sets of compounds as desired. The synthetic schemes for such reactions is shown in FIGS. 2 and 3. The structures of the resulting fluorophores have been confirmed by nuclear magnetic resonance spectroscopy and mass spectrometry, and certain fluorophore structures have been confirmed by X-ray crystallography.

Current methods of quantifying Pb₂₊ in samples may require expensive instrumentation, are not readily portable and are restricted to in vitro measurement. The presently disclosed compounds, lead sensors and methods provide cost effective, portable, rapid and reliable methods for detecting and quantifying lead content in gunshot residue samples that are also highly sensitive and selective for lead over other metal ion contaminants. In addition, the lead binding fluorophore is water soluble and also may be cell permeable.

The lead ion sensor fluorophore according to the invention has a structure represented in its protected form by Formula II below.

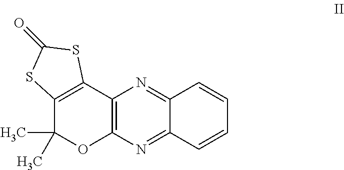 [SMILES: CC1(C)Oc2nc3ccccc3nc2-c2sc(=O)sc21]

As mentioned herein, the Formula II may be also be represented by Formula I, wherein X is O, R¹ and R² are each methyl, and R³ and R⁴ come together to form a benzo ring. The structure represented by Formula II may be hydrolyzed, for example by acid or base hydrolysis, including, for example, hydrolysis with Et₄NOH, to form the active fluorescent binder which binds to Pb²⁺ to form a highly fluorescent complex. The active fluorophore compound is believed to have a structure represented by Formula III:

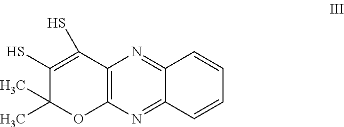 [SMILES: CC1(C)Oc2nc3ccccc3nc2C(S)=C1S]

or the corresponding thiolate compound (i.e., where one or both thiols of the ene-dithiol are deprotonated). In the presence of Pb²⁺ ions and a hydrolyzing agent, the dithiocarbonate functionality of compound II may be hydrolyzed and the resulting fluorescent binder compound binds with the Pb²⁺ to form a Pb²⁺/binder complex. According to specific embodiments, the fluorescent binder of the present disclosure has a high sensitivity for Pb²⁺ ions, even in the presence of other metal ions. For example, in one embodiment, the fluorescent binder has a sensitivity for Pb²⁺ as measured by apparent dissociation coefficient, K_{d} (as determined by measuring the fluorescence of the complex) that may be less than 500 nanomolar (nM). In other embodiments, the K_{d} may be less than 300 nM, or in certain embodiments less than 250 nM. In specific embodiments, the fluorescent binder may have a sensitivity for Pb²⁺ at K_{d}=217 nM at pH=10. As will be understood by one of ordinary skill in the art, the K_{d} for binding of Pb²⁺ may vary according to the conditions of the experiment, including, but not limited to solvent, temperature, pH, etc.

When the fluorescent binder according to the present embodiments binds with Pb²⁺ to form a Pb²⁺/binder complex, the complex fluoresces with a high optical brightness. For example, according to the present embodiments, the unbound fluorescent binder represented by Formula III (or Formula II, in its protected form) may have an excitation band with a absorbance maximum λₘₐₓ at a wavelength centered around 415 nm, an emission band with a emission maximum λₘₐₓ centered around 465 and a quantum yield for the fluorescence emission of φ=0.12. When the fluorescent binder is bound to Pb²⁺ ions, the resulting Pb²⁺/binder complex may have an excitation band with a λₘₐₓ centered around 389 nm, an emission band with a λₘₐₓ centered around 423 nm, and a quantum yield of φ=0.63. Quantum yields were calculated with reference to the quantum yield of fluorescein (φ=0.95). As reported herein, the wavelength band for excitation and emission of the unbound fluorescent binder and the Pb²⁺/binder complex may have excitation and emission Amax values that vary by plus or minus 50 nm (i.e., the unbound fluorescent binder may have an excitation band with a λₘₐₓ value ranging from 365 nm to 465 nm and an emission band with a λₘₐₓ value ranging from 415 nm to 515 nm; and the bound fluorescent binder complex may have a excitation band with a λₘₐₓ value ranging from 339 nm to 439 nm and an emission band with λₘₐₓ value ranging from 373 nm to 473 nm).

According to certain embodiments, the fluorescent binder discussed herein selectively binds to Pb²⁺ over other metal ions including other transition metal ions. According to various embodiments, the fluorescence emission intensity of the Pb²⁺/binder complex is greater than a fluorescence emission intensity of other metal ion/binder complexes. Other metal ions that may form a metal ion/binder complex that has a lower fluorescence emission intensity than that of the Pb²⁺/binder complex include, but are not limited to Li⁺, Na⁺, K⁺, Ca²⁺, Mg²⁺, Co²⁺, Ni²⁺, Cu²⁺, Zn²⁺, Cd²⁺, Mn²⁺, Hg²⁺, Sn²⁺, As³⁺, and mixtures thereof. For example, according to one non-limiting embodiment, the Pb²⁺/binder complex in the presence of the other metal ions may have a fluorescence emission intensity at least about 10 times greater than the fluorescence emission intensity of another metal ion/binder complex. In other embodiments, the Pb²⁺/binder complex in the presence of the other metal may have a fluorescence emission intensity at least about 20 times greater than the fluorescence emission intensity of another metal ion/binder complex. Since the Pb²⁺/binder complex fluoresces with a significantly greater intensity than other metal ion/binder complexes, the fluorescent binder serves as a selective detector for lead ions in various samples, including samples that may contain other metal ions.

The present disclosure provides a Pb²⁺ sensor for Pb²⁺ ions in a sample analyzed for GSR. The Pb²⁺ sensor comprises a matrix material and a fluorophore represented in its protected form by Formula II, wherein the fluorophore is dissolved in, embedded in, affixed in, absorbed in, or suspended in the matrix material and forms a fluorescent complex when bound in its unprotected form (represented by Formula III) to Pb²⁺. As used herein, the terms “fluorescent binder” and “fluorophore” refer to a compound having a structure represented by Formula II (in the fluorophore's protected form) or Formula III. According to specific embodiments, the Pb²⁺ sensor is selective for Pb²⁺ ions over other metal ions, such as, but not limited to metal ions selected from Li⁺, Na⁺, K⁺, Ca²⁺, Mg²⁺, Fe²⁺, Co²⁺, Ni²⁺, Cu²⁺, Zn²⁺, Cd²⁺, Mn²⁺, Hg²⁺, Sn²⁺, As³⁺, and mixtures thereof. In addition, the Pb²⁺/fluorophore complex may have a greater fluorescence emission intensity compared to the fluorescence emission intensity of complexes between the fluorophore in other metal ions.

The fluorescence emission intensity of the Pb²⁺/complex in a GSR sample may be measured and compared to a standard calibration plot or values to determine the Pb²⁺ ion concentration in the sample. The concentration level can contribute to the investigation of an accident or crime scene and to determining the person who fired the firearm. For example, if the intensity is significant, it can be ascertained that the sample correlates to a person that filed the firearm and if the intensity is less significant, it can be ascertained that the sample is a result of secondary transfer and therefore, the person to which the sample correlates was in proximity to the discharged firearm or the person who fired the firearm. That is, a GSR sample obtained troy the person who fired the firearm will have a higher concentration of Pb²⁺ ions than a GSR sample obtained from a person that was only near the firearm when it was discharged.

The matrix material may be any material suitable for dissolving, embedding, affixing, absorbing, or suspending the fluorophore that can be used to test a sample composition for Pb²⁺ ion concentration. For example, the matrix material may be, but is not limited to, a material selected from the group consisting of an aqueous solvent, a gel, a sol-gel material, a solvent, a paper, a polymer, a nanoparticle, a solid state material, and a surface modified material. One having ordinary skill in the art will recognize that other matrix materials may be used without departing from the intent of the invention as described herein.

In one embodiment, a Pb²⁺ sensor may be immobilized in the form of a gel. For example, according to one embodiment, the gel may be prepared using sol-gel technology from an appropriate starting material. It may be important to have a matrix material with minimal absorption in the regions in which the fluorophore or the Pb²⁺/fluorophore complex absorb or emit light. Suitable materials for gels may include gels based on silicon and aluminum. Such gels may be produced in numerous ways and methods of preparation of such gels are known in the art. For example, hydrolysis of silicon-alkoxide in the presence of an alcohol would produce a suitable gel material. Common precursors for preparing silica based gels include, but are not limited to, tetramethoxysilane (Si(OCH₃)₄) and tetraethoxysilane (Si(OC₂H₅)₄) and the corresponding alcohols. In addition, various silica based gel materials are commercially available with particle sizes ranging from 10 nm to 40 nm and are known to those of ordinary skill in the art. In certain embodiments, the fluorophore may be doped into the gel to produce Pb²⁺ sensors of the present disclosure. Gel materials may also be made having differentially shaped and sized particles, which may be doped with the fluorophore to provide versatile devices. One critical component of the functioning of the get may be the response time, which can, at least in part, be controlled by manipulating the porosity of the gel. The porosity of the gel may be controlled, for example, by the method of gel preparation using methods reported in the literature and known to those of ordinary skill. According to one embodiment, the gel may be engineered to have functionality which may be reactive with a functionalized fluorophore and thus can chemically or physically link the fluorophore to the structure of the gel. For example, the functionalized fluorophore may be polymerized with the gel and thus, incorporated into the structure of the gel.

Another embodiment may include a lead sensing paper, analogous to a pH paper. According to this embodiment, a known concentration of fluorophore may be impregnated into a porous paper. The fluorophore impregnated paper may then be contacted with a GSR sample comprising Pb²⁺ ions and the paper would fluoresce with an emission intensity, as determined by a fluorophoric device, that may correspond to the Pb²⁺ ion concentration in the composition.

In general, the combined matrix material and fluorophore is placed in contact with a sample analyzed for GSR. In certain embodiments, the sample may be contact with the matrix/fluorophore without need to collecting a portion of the sample from the surface or the substrate on which the sample was originally deposited. In other embodiments, the sample is collected from the surface or substrate and the collected sample is contacted with the matrix/fluorophore. If, as a result of the contact, the sample fluoresces, it is further concluded that the sample contains a concentration of Pb²⁺ ions and therefore, it is further concluded that the sample is composed of GSR. In addition to determining the mere presence of Pb²⁺ ions and GSR in the sample, the emission intensity of the fluoresce can be assessed to determine the level of Pb²⁺ ion concentration in the sample, which can provide additional information about the GSR sample and its origin, e.g., resulting from direct transfer to the person firing the weapon or from secondary transfer to a person located near the weapon or from a person in contact with the person who fired the weapon.

In specific embodiments of the Pb²³⁰ sensor, the sensor may further comprise a device capable of measuring the intensity of the fluorescence emission spectrum of the Pb²⁺/fluorophore complex in the matrix material. Examples of devices include, but are not limited to, fluorophoric devices and spectrometers, such as fluorescence spectrometers or fluorometers, laser fluorescence spectrometers, and the like. The fluorescence emission spectrum may be compared to emissions of known standards, for example a calibration plot, to determine the concentration of Pb²⁺ in the sample. For certain embodiments, the determination of the Pb²⁺ may be automated, such as by use of a computer, sampler, or other electronic device. For example, the computer or other electronic device may compare the fluorescence emission spectrum with the spectra of standards and determine the Pb²⁺ ion concentration in the sample. In other embodiments, the sample and standard spectra may be compared by a user of the sensor and a Pb²⁺ ion concentration of the sample may be determined based on the emission intensity of the Pb²⁺/complex.

It is contemplated that the present disclosure provides for portable devices, such as kits, that are effective for on-site testing, detection and analysis of particle-containing samples for the presence of lead ions and to provide a determination of GSR. The term “on-site” generally refers to locations outside of a laboratory, such as, the location or scene where the firearm was discharged, such as, where an accident occurred or a crime was committed. The portable devices in accordance with the invention employ analytical means that do not require complex analytical instruments and procedures, which are typically employed in a forensic laboratory. Thus, the devices or kits of the invention provide the capability to determine the presence or absence of GSR in a particle-containing sample on-site without the need to collect a portion of the sample from the scene and transport it to a laboratory for analysis.

For example, field kits may be provided to police officers and/or crime scene investigators such that a presumptive test for GSR can be performed in the field prior to providing a SEM stub for confirmation. The analysis can be useful in eliminating negative samples from being analyzed in the more instrument-intensive confirmatory method that is performed in the laboratory by a qualified forensic scientist.

The devices or kits of the invention include a fluorophore sensor material or compound. The fluorophore compound forms a complex with lead ions and the fluorophore/lead complex fluoresces with an intensity greater than complexes formed by the fluorophore with other metals. Thus, according to the invention, the fluorophore compound in the device or kit is contacted with a particle-containing sample to he analyzed (which is suspected of containing GSR). A fluorescent light source is then applied to illuminate the sample with fluorophore compound applied thereto. It is contemplated that the fluorescent light source is contained within the device or kit. Visual observation is performed to assess whether fluorescence occurs. If the sample includes the presence of lead, the fluorophore compound will complex with the lead ions in the sample, form a fluorophore/lead complex, and fluoresce. Thus, fluorescence of the fluorophore/lead complex will be evident to show the presence of lead in the sample and therefore, also will be effective to conclude that the sample contains GSR. In contrast, if the sample does not include lead, the fluorophore compound will not he able to complex with any lead ions in the sample, a fluorophore/lead complex will not form and will not fluoresce. Thus, the kick of fluorescence will be evident to show the absence of lead in the sample and therefore, also will be effective to conclude that the sample does not contain gunshot residue.

The kit does not rely on the presence of electrical power and/or computers. The kit can be used by a person that is not trained or skilled in forensic science. In general, the kit typically includes a package with one or more containers or chambers (e.g., bottle, plate, tube, and dish) having therein specific materials. The kit preferably has directions for using the individual materials contained in any one of individual containers constituting the kit. In certain embodiments, the kit of the invention is a package which includes the fluorophore sensor material or compound and a fluorescent light source. In addition, tools may be included in the kit for collecting a sample to be analyzed and a container or chamber in which to carry out the analysis of the sample using the fluorophore compound. The “directions” for using the kit may be written on a paper or other kind of a medium or may be printed out. Alternatively, the “directions” may be in the form of a magnetic tape or an electronic medium such as a computer-readable disc or tape and a CD-ROM.

The fluorophore sensor compound may be in various forms. For example, the fluorophore sensor compound be in the form of a liquid such that the liquid is applied to the sample to be analyzed. The fluorophore may be applied to directly to the particle-containing sample (as-found) or a portion of the sample may be collected, placed in a vessel or container and then contacted with the fluorophore compound. It is also contemplated that the fluorophore compound may be in a container or holder and the sample added to the container of fluorophore compound. An adequate period of time may be needed to allow for a reaction to occur; e.g., a few seconds or a few minutes. The resulting sample with fluorophore compound is examined for fluorescence. The observation can be made by the naked eye or alternatively, by use of an instrument. If fluorescence occurs, a comparison with a chart, e.g., a color chart, may be conducted to determine the degree or level of lead/GSR in the sample or alternatively, an instrument may be used to obtain a reading. It is contemplated that any instruments needed for observation or comparison will be included in the kit.

In certain embodiments, a chamber provided within the kit can accommodate a reaction mixture and, if needed, may include a heating element. The reaction mixture includes the sample to be analyzed and the fluorophore sensor material or compound. The heating element can heat the reaction mixture to a temperature that is sufficient to form the lead fluorophore complex.

The kit can further include an ultraviolet light excitation source to excite the lead/fluorophore complex and to produce a fluorescence light output. In certain embodiments, an optical output filter is operable to present the fluorescence light output in a predetermined bandwidth of wavelength. A photon sensing detector captures and converts the fluorescence light output to an electrical signal. An electrical signal detection and amplification circuit transmits and displays a measurement of the fluorescence light output to a wireless device.

It will be appreciated that kits in accordance with the invention may include alternate designs and configurations.

The measurement of the fluorescent light output in the sample to be analyzed can be compared with a measurement of fluorescent light output from a non-GSR or non-lead-containing sample, e.g. a control sample, to detect the presence or absence of the of lead/GSR in the sample. If the measurement of the fluorescent light output in the sample has intensity greater than the measurement of the fluorescent light in the control sample, the presence of lead/GSR is detected. If the measurement of the fluorescent light output in the sample has intensity comparable with, e.g., not measurably greater than, the measurement of the fluorescent light in the control sample, it is determined that the lead/GSR is absent or its concentration is below a predetermined detection limit.

In certain embodiments, the method of the invention includes obtaining a particle-containing sample from a location where a firearm has been discharged, e.g., a crime scene or an accident scene; preparing a reaction mixture including combining the sample with a fluorophore sensor material or compound; optionally heating the reaction mixture to a temperature sufficient to bind the fluorophore compound and lead ions in the sample to form a lead/fluorophore complex; exciting the lead/fluorophore complex by employing a ultraviolet light excitation source; producing a fluorescent light output; optionally filtering the fluorescent light output using an optical output filter; optionally capturing the fluorescent light output and cornering the captured fluorescent light output to an electrical signal using a photon sensing detector; and optionally amplifying and displaying a measurement of the fluorescent light output on a wireless device.

The particle-containing sample to be analyzed can be obtained using a variety of conventional techniques and the techniques, employed are not critical to the invention.

Further embodiments of the present disclosure provide methods for detecting Pb²⁺ ion concentrations, for example the Pb²⁺ ion concentration in a sample. According to one embodiment, the methods may comprise contacting a Pb sensor comprising a fluorophore represented in its protected form by Formula II, as described in detail herein with reference to the fluorescent binder, with a composition, wherein at least a portion (and in certain embodiments all or substantially all) of the Pb²⁺ ions in the composition binds with the fluorophore in its unprotected form (represented by Formula III) to form a complex; and measuring a fluorescence emission intensity of the complex. As used herein, the term “substantially all” when used in reference to metal ion binding means an amount of metal ions equivalent to the concentration of the metal ion/binder complex as determined by the equilibrium expression for the reaction/complexation of the metal ion with the fluorophore. As described herein, the complex may have a fluorescence emission intensity that is greater than the fluorescence emission intensity of a complex formed from the fluorophore binding with another metal, such as, but not limited to, the other metals described herein. In specific embodiments, the method may be used to selectively detect Pb²⁺ ions in the composition in the presence of other metal ions.

In certain embodiments, the method may further comprise irradiating the complex with electromagnetic radiation having a wavelength(s) equal to the excitation wavelength or band of the Pb²⁺/fluorophore complex, as described herein. In specific embodiments, the method may further comprise quantifying the lead ion concentration by calculating a concentration of Pb²⁺ ions in the composition based on the fluorescence emission intensity of the Pb²⁺/fluorophore complex. For example, the fluorescence emission intensity, as determined by the fluorescence emission spectrum, may be compared to a standard fluorescence emission calibration plot for the Pb²⁺/fluorophore complex to determine the Pb²⁺ ion concentration in the composition.

According to specific embodiments of the methods herein, the Pb²⁺ sensor may further comprise a matrix material, such as the matrix materials described in detail herein, wherein the fluorophore may be dissolved in, embedded in, affixed in, absorbed in, or suspended in the matrix material. As discussed herein, the fluorophore may be modified by replacing one or more hydrogens on the fluorophore with a group reactive with a functionality in the matrix material. According to specific embodiments, the fluorophore may be linked to or otherwise attached to the matrix material.

There are various other advantages associated with the methods and devices of the invention which include, but are not limited to, the ability to quantify the gunshot residue in an analyzed sample. It is typical for known analytical methods and devices to merely identify the presence or absence of gunshot residue without the ability to determine the specific amount of gunshot residue present. Furthermore, it is contemplated that the methods and devices of the invention may be effective to determine the type, of ammunition used, the distance of the weapon when fired from the surface of interest, and how many shots were fired in a specific location.

Another added benefit of this method is that it allows for more quantitative studies on the science of GSR. Secondary transfer studies are based on the number and size of the particles found that are characteristic for GSR. However, it is known that GSR also contains particles that are not necessarily classified as characteristic of GSR, but still contain lead. Use of the lead ion sensor in accordance with the invention for analyzing GSR, allows one to directly quantitate the total amount of lead collected, and this measurement may be used as a relative indication of the amount of GSR. This information is highly useful, as it may be useful in responding to the following questions that remain unanswered based on known SEM/EDS analysis:

Is there a significant difference between primary and secondary transfer gunshot residue?

Can it be determined/estimated how far away the weapon was from the surface of deposition?

Is it possible to determine how many shots wet e fired when traditionally indicative objects are not available, i.e. bullets, casings?

While various specific embodiments have been described in detail herein, the present disclosure is intended to cover various different combinations of the disclosed embodiments and is not limited to those specific embodiments described herein. Various embodiments of the present disclosure will be better understood when read in conjunction with the following non-limiting Examples. The procedures set forth in the Examples below are not intended to be limiting herein, as those skilled in the art will appreciate that various modifications to the procedures set forth in the Examples, as well as to other procedures not described in the Examples, may be useful in practicing the invention as described herein and set forth in the appended claims.

### EXAMPLES

### Example 1

A preliminary experiment was conducted to test Whether the presence of barium in GSR would inhibit the probe's response to lead. Standards were prepared. Equal amounts of lead and barium were added to two aliquots of a lead sensor according to Formula II. Also, a third sample containing both metals was prepared by first tickling barium, then lead. Fluorescence was measured on a Horiba Fluoromax-4. All three solutions emitted at the same wavelength. Although, the intensity of the free ligand without any added metal was comparable to that of the ligand plus barium. The intensity, however, increased in the presence of lead. The addition of baruim to both the lead complex and the free ligand seemed to slightly increase intensity. It was considered that this may be a trend or simply due to error. Other metals commonly found in GSR, such as iron, have already been shown not to interfere with the fluorescent response of the lead complex.

### Example 2

A preliminary experiment was conducted wherein an extraction of a GSR sample was used for a complexation reaction. A gun was fired with the barrel held to a piece of paper into the ground. A visible, dark grey residue was left on the paper. A cutting of the paper was taken and placed in a test tube with concentrated acid. The tube was vortexed in order to allow the paper to shed the residue. The fibers from the paper were filtered out, and the acid was neutralized with base before adding the GSR extraction to a solution of lead sensor according to Formula II. Fluorescence was measured. Preliminary studies showed that there would be no interference from other metals in the mixture present in GSR. Direct testing with an actual GSR extraction yielded positive results.

### Example 3

Two volunteers were selected to participate in this study. Gunshot residue samples were collected at an indoor shooting range. Each of the volunteers wore clean nitrile gloves, discharged a firearm twice, and returned to a sampling station to be swabbed. The firearms used in this study included a 9-mm glock pistol and a 22 caliber revolver. Samples from the glove on each hand were collected via swabs wetted with 5% HNO₃, stored in clean screw-top test tubes and transported back to the laboratory for analysis. The volunteers were instructed to put on a clean pair of gloves for each test. Controls included swabs from both hands of a volunteer who put on clean gloves, left the sampling station, entered the firing range and then returned to the sampling station without handling or discharging a firearm. A blank swab was also taken at the firing range as a control.

To each swab-containing tube, 1,200 mL deionized water was added and the tubes were vortexed for 30 seconds. The resulting aqueous solution was decanted from the collection tubes into new microcentrifuge tubes. From each sample, 300 μL was taken and diluted to 10 mL with 5% HNO₃. These diluted solutions were tested for the following metals using ICP-MS analysis: ²⁷Al, ²⁸Si, ¹¹⁸Sn, ¹²¹Sb, ¹²⁸Ba, and ²⁰⁸Pb. The remaining sample was used for fluorescent analysis.

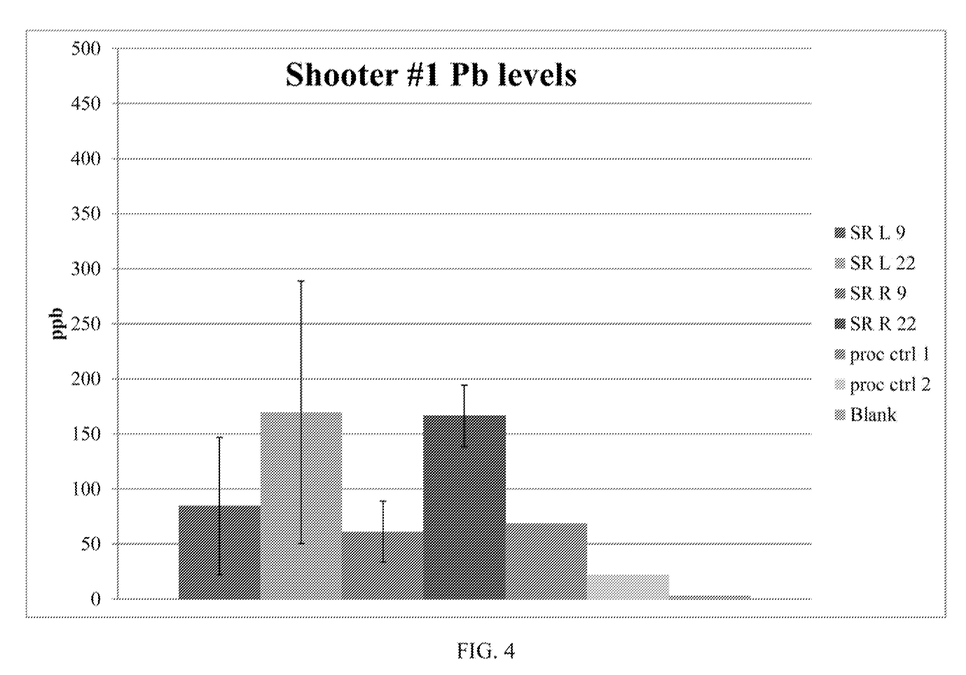

Standard solutions of Pb²⁺ in water were prepared in the following concentrations: 5, 10, 20, 30, 40, and 50. A calibration curse was prepared using these standards. For all standards, 500 μL of solution was added to a solution of 1 μM lead sensor according to Formula II (designated “LG”), 2 μM Et₄NOH in 2.5% MeOH and water. The results are shown in the below Tables 1 and 2, and FIGS. 4, 5, 6, 7 and 8.

TABLE 1
Normalized Calibration Curve for LG standards, intercept
set to (0,0)
GSR Quick Extract Measurements

| | Emission | | | Total amount |
|---|---|---|---|---|
| | Intensity | Normalized | Calculated | of Pb in |
| Test | (CPS) | Intensity | concentration (ppb) | extract (ng) |
| 1 | 1.568 × 10⁶ | n/a | Outside range of | n/a |
| | | | quantitation | |
| 2 | 1.163 × 10⁶ | 1.992 × 10⁵ | 34.6 ± 2.7 | 17.3 ± 1.35 |
| 3 | 1.239 × 10⁶ | 2.752 × 10⁵ | 47.8 ± 2.9 | 23.9 ± 1.45 |

TABLE 2
ICP-MS Verification

| | | | | | | |
|---|---|---|---|---|---|---|
| ID | A127 | Si28 | Sn118 | Sb121 | Ba138 | Pb208 |

Concentration μg/l (ppb)

| | | | | | | | |
|---|---|---|---|---|---|---|---|
| AD, L, 9 | A | 837 | 1.36 | 26.8 | 4.3 | 1.79 | 151 |
| 338.5^{a} | B | −80.4 | 1.36 | 157 | 13.7 | 1.79 | 371 |
| 141.5^{b} | C | 1110 | 1.36 | 68.4 | 8.3 | 1.79 | 339 |
| | D | 1240 | 1.36 | 36.2 | 11.3 | 1.79 | 493 |
| SR, L 9 | E | 1140 | 1.36 | 50.2 | 9.12 | 1.79 | 177 |
| 84.5^{a} | F | 557 | 112.57 | 3.49 | 1.07 | 204 | 55.1 |
| 62.4^{b} | G | 403 | 81.77 | 2.58 | 0.882 | 254 | 41.2 |
| | H | 336 | 78.17 | 2.24 | 1.13 | 305 | 64.7 |
| AD R, 9 | I | 861 | 1.36 | 39.7 | 7.74 | 1.8 | 209 |
| 119.125^{a} | J | 448 | 128.57 | 1.99 | 2.73 | 317 | 93.7 |
| 60.6^{b} | K | 582 | 1.36 | 15.8 | 1.67 | 345 | 76.4 |
| | L | 440 | 1.36 | 2.89 | 2.37 | 1.8 | 97.4 |
| SR, R 9 | M | 189 | 0.86 | 0.834 | 0.487 | 147 | 22.6 |
| 61.2^{a} | N | 448 | 196.57 | 12.4 | 1.31 | 240 | 66.2 |
| 27.7^{b} | O | 386 | 1.36 | 2.37 | 1.99 | 364 | 67.3 |
| | P | 420 | 1.36 | 23.8 | 6.07 | 284 | 88.6 |
| AD L, 22 | Q | 818 | 1.36 | 27.7 | 3.93 | 387 | 250 |
| 315.5^{a} | R | 455 | 1.36 | 3.63 | 0.662 | 1.8 | 425 |
| 75.8^{b} | S | 363 | 1.36 | 27 | 1.21 | 346 | 293 |
| | T | 403 | 1.36 | 10.7 | 1.06 | 196 | 294 |
| SR L 22 | U | 395 | 130.57 | 1.96 | 0.362 | 167 | 62.8 |
| 169.6^{a} | V | 539 | 1.36 | 20.3 | 0.895 | 308 | 213 |
| 119.1^{b} | W | 448 | 1.36 | 21.9 | 1.17 | 187 | 318 |
| | X | 533 | 208.57 | 3.18 | 0.321 | 245 | 84.7 |
| AD R 22 | Y | 767 | 1.36 | 23 | 4.09 | 275 | 259 |
| 265.8^{a} | Z | 444 | 83.07 | 7.14 | 1.26 | 212 | 337 |
| 90.7^{b} | AA | 328 | 83.47 | 8.1 | 0.844 | 258 | 327 |
| | BB | 459 | 201.57 | 2.11 | 0.638 | 194 | 140 |
| SR R 22 | CC | 452 | 1.36 | 11.8 | 0.412 | 269 | 187 |
| 166.3^{a} | DD | 330 | 1.36 | 2.36 | 0.462 | 312 | 152 |
| 27.9^{b} | EE | 270 | 1.36 | 8.32 | 0.448 | 231 | 134 |
| | FF | 236 | 0.86 | 1.84 | 0.239 | 134 | 192 |
| SR procedure control | GG | 686 | 1.36 | 15.7 | 3.1 | 345 | 68.7 |
| SR procedure control | HH | 430 | 99.17 | 2.82 | 0.327 | 234 | 22.4 |
| Blank swab | II | −80.4 | 1.36 | 23 | 383 | 1.8 | 3.24 |
| surface swab | JJ | 360 | 85.97 | 2.07 | 3.44 | 98.9 | 88.3 |

^{a}Average within set of four repeats
^{b}Standard deviation of average

FIG. 4 shows a summary of lead amounts of shooter 1 (ID; SR), left (L) and right (R) hands after firing a 9 (pistol) or 22 (revolver) caliber firearm as well as controls. The error bars signify standard deviation.

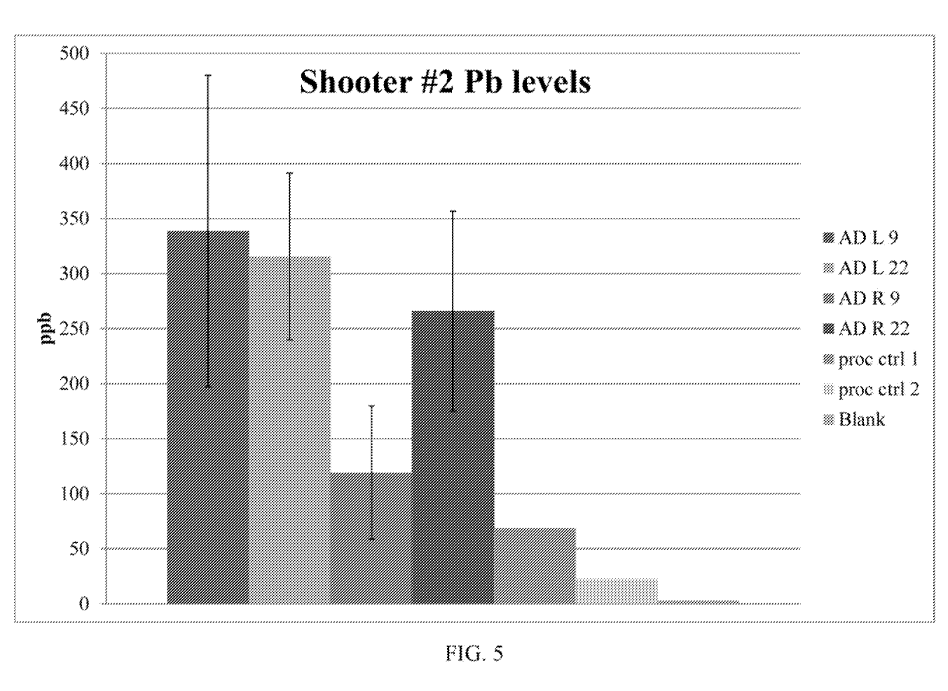

FIG. 5 shows a summary of lead amounts of shooter 2 (ID: AD), left (L) and right (R) hands after firing a 9 (pistol) or 22 (revolver) caliber firearm as well as controls. Error bars signify standard deviation.

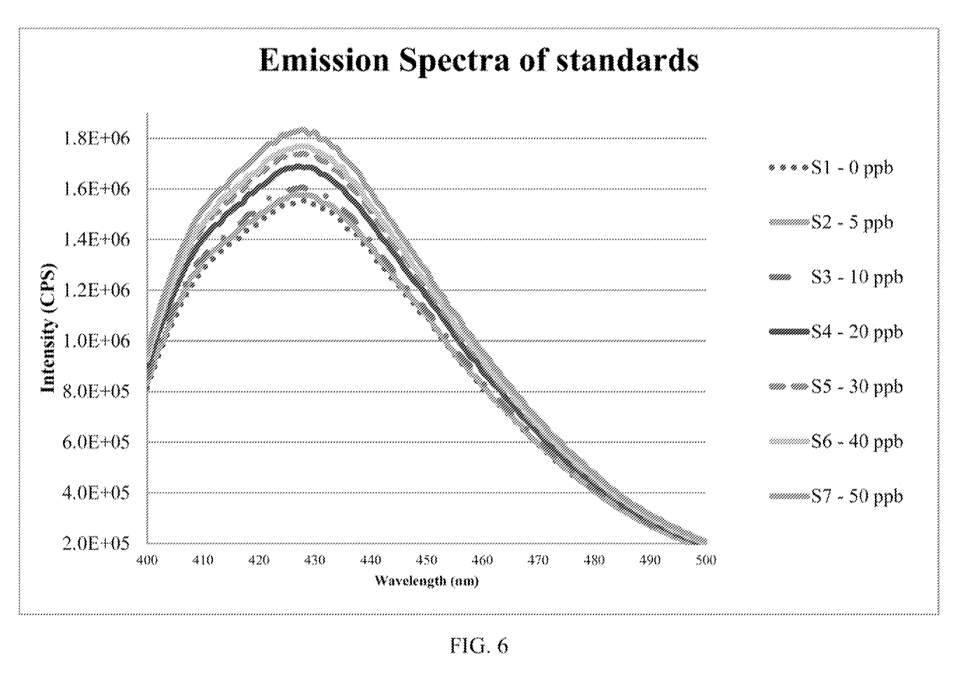

FIG. 6 shows emission spectra of standard lead solutions upon addition to LG at the designated concentrations.

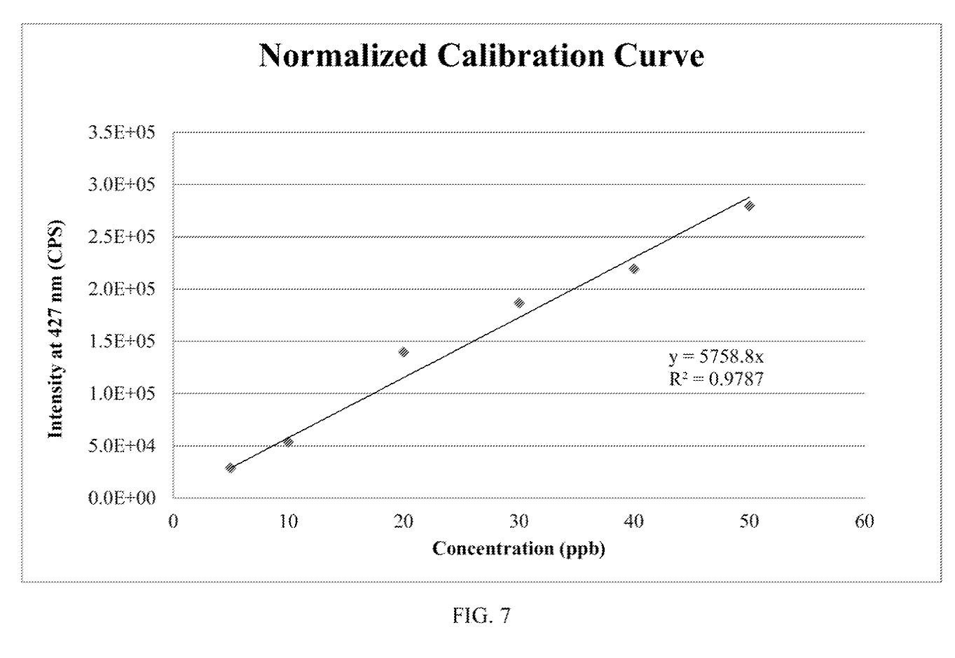

FIG. 7 shows a plot of maximum emission intensity (λ=426 nm) after subtraction of the blank (0 ppb Pb). m=5758.8, r²=0.9787

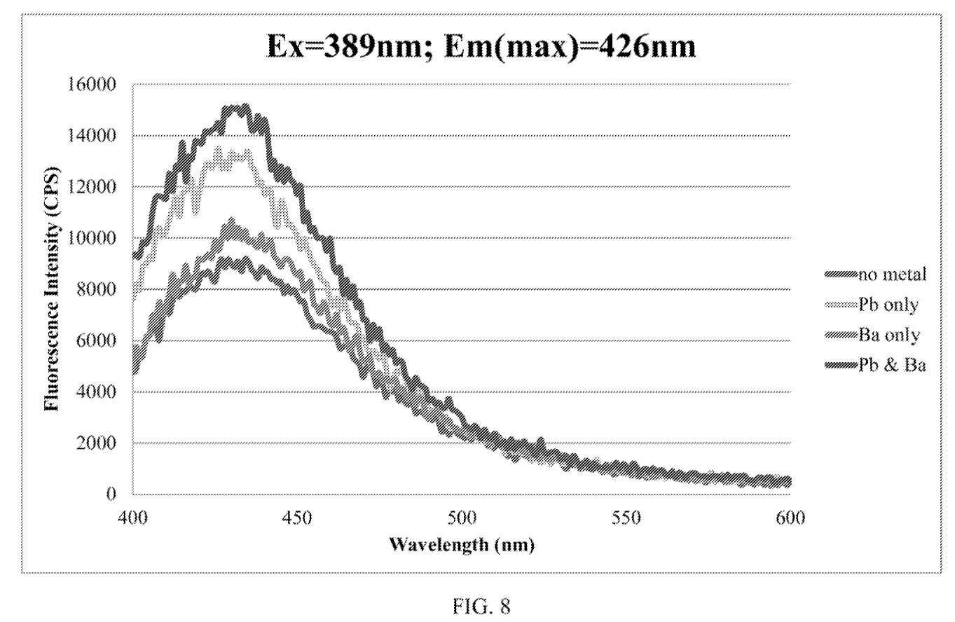

FIG. 8 shows fluorescence spectra of samples containing equal amounts of LG added to: Pb only, Ba only, Pb and Ba, or water.

The invention has been described in detail in the foregoing embodiment for the purpose of illustration, it is to be understood that such detail is solely for that purpose and that variations can be made therein by those skilled in the art without departing from the spirit and scope of the invention.

We claim:

1. A Pb²⁺ sensor for analyzing a particle-containing sample for gunshot residue, the sensor comprising:
a matrix material; and
a fluorophore represented in its protected form by Formula II: 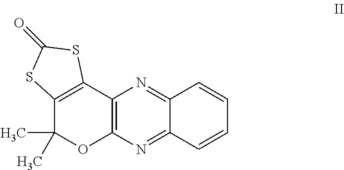
wherein, the fluorophore is dissolved in, embedded in, affixed in absorbed in, or suspended in the matrix material and forms a fluorescent complex when bound in its unprotected form to Pb²⁺.

2. The Pb²⁺ sensor of claim 1, wherein the matrix material comprises a material selected from the group consisting of an aqueous solvent, a gel, a sol-gel material, a solvent, a paper, a polymer, a nanoparticle, a solid state material, and a surface-modified material.

3. The Pb²⁺ sensor of claim 1, further comprising a device capable of measuring an intensity of a fluorescence emission spectrum.

4. The Pb⁺ sensor of claim 1, wherein one or more of the hydrogens on the fluorophore is replaced with a group reactive with a functionality in the matrix material.

5. A method of analyzing a gunshot residue, comprising:
contacting a particle-containing gunshot residue sample with a Pb²⁺ sensor comprising a fluorophore represented in its protected form by Formula II: 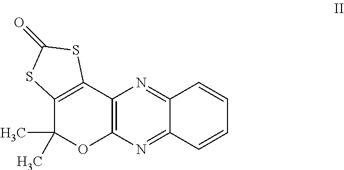
wherein, Pb²⁺ in the gunshot residue sample binds with the fluorophore in its unprotected form to form a complex; and
determining a presence or absence of fluorescence of the sample,
wherein, the presence of fluorescence correlates to the presence of Pb²⁺ ions and the absence of fluorescence correlates to the absence of Pb²⁺ ions.

6. The method of claim 5, further comprising measuring a fluorescence emission intensity of the complex.

7. The method of claim 5, wherein the method selectively detects Pb²⁺ in the presence of other metal ions.

8. The method of claim 7, wherein the fluorescence emission intensity of the complex formed with Pb²⁺ is greater than the fluorescence emission intensity of a complex formed from the fluorophore binding with another metal,

9. The method of claim 6, further comprising calculating a concentration of Pb²⁺ ions in the gunshot residue sample based on the fluorescence emission intensity of the complex.

10. The method of claim 5, further comprising:
establishing a threshold value based on one of fluorescence emission intensity of the complex or concentration of the Pb²⁺ ions;
comparing one of fluorescence emission intensity of the complex or the concentration of Pb²⁺ ions for the gunshot residue sample with the threshold value;
determining that the gunshot residue sample is a result of a direct transfer if the threshold value is met or exceeded; and
determining that the gunshot residue sample is a result of a secondary transfer if the threshold value is not met.

11. A portable device for detecting Pb²⁺ ions in a particle-containing sample for analysis of gunshot residue comprising:
a fluorophore sensor; and
an activation source to excite a complex formed by the Pb²⁺ ions and the fluorophore sensor material and to produce a fluorescence light output.

12. A portable method of analyzing a gunshot residue sample, comprising:
transporting a kit to a site of a discharged firearm, the kit comprising:
a fluorophore sensor material; and
an illumination source;
obtaining a gunshot residue sample to be analyzed;
contacting the sample, with the fluorophore sensor material;
applying the illumination source to the sample with the fluorophore sensor material;
visually observing whether the sample with the fluorophore sensor material fluoresces;
determining a presence of Pb²⁺ ions wherein the sample fluoresces based on visual observation; and
determining an absence of Pb² ions wherein the sample does not fluoresce based on visual observation.
